(19) United States
(12) Patent Application Publication
(10) Pub. No.: US 2007/0224694 A1
(43) Pub. Date: Sep. 27, 2007

(54) Method and system for hyperspectral detection of animal diseases

(76) Inventor: Daniel Matthew Puchalski, Colorado Spring, CO (US)

Correspondence Address: HOLLAND & KNIGHT LLP, 2099 PENNSYLVANIA AVE, N.W., WASHINGTON, DC, 20006, US

(21) Appl. No.: 11/350,776

(22) Filed: Feb. 10, 2006

(51) Int. Cl. G01N 21/62; G01J 3/00; A61B 5/05

(52) U.S. Cl. 436/171; 356/300; 600/407

(57) ABSTRACT

An entirely new application for hyperspectral data has been identified as well as an entirely new means to detect TSE related diseases. This application can be applied to any disease where foreign matter builds in an observable location such as central nervous system tissue (retinal nerves). By using imagable central nervous system tissue, malformed proteins may now be detected via a hyperspectral scanner and the application of hyperspectral technology to detect disease via eye tissue.

### FIELD OF THE INVENTION

The present invention relates to the medical imaging, disease detection and hyperspectral industries.

### BACKGROUND OF THE INVENTION

Bovine Spongiform Encephalopathy (BSE or Mad Cow Disease) is caused by a malformed prion protein. The three areas of high infectivity according to the World Health Organization consist of the central nervous system tissues; the brain, spinal and eye tissues. The only means approved by the United States to detect BSE is immunohistochemistry, which performed post mortem on the brain tissue. The brains are sectioned, stained for structure and with an antibody to highlight the prion protein then examined under a microscope for the telltale spongiform morphology. Other non-FDA approved methods to detect include:
Bio Assay—The most sensitive test, which is also the lengthiest, is the mouse bioassay, in which suspect brain tissue is injected into the brain of a mouse, and 6 months to 1 year later, the mouse is killed and its brain examined to see if it has developed disease.
Immunoblotting—Immunologic methods for isolating and quantitatively measuring immunoreactive substances. When used with immune reagents such as monoclonal antibodies, the process is known generically as western blot analysis (blotting, western). It allows one to visualize antibodies directed against each viral protein . . . proteins are electrophoresed into a gel. As the proteins migrate through the gel they are separated based upon size and charge. Characteristically, smaller proteins migrate through the gel faster than larger proteins.
ELISA—Enzyme-Linked Immunosorbent Assay, sometimes called an enzyme immunoassay (EIA) is the first and most basic test to determine if an individual is positive for a selected pathogen. “This is a rapid immunochemical test that involves an enzyme (a protein that catalyzes a biochemical reaction). It also involves an antibody or antigen (immunologic molecules). ELISA tests are utilized to detect substances that have antigenic properties, primarily proteins (as opposed to small molecules and ions such as glucose and potassium). Some of these include hormones, bacterial antigens and antibodies.” http://www.medterms.com/script/main/art.asp?articlekey=9100

### SUMMARY OF THE INVENTION

We believe there is a better way to detect the presence of these malformed prion proteins with application to the family of diseases called transmissible spongiform encephalopathy (TSE).

When light (or other energy) is absorbed by an atom, an electron jumps from a low energy orbital to a higher energy orbital. The electrons jumping between different orbitals produce the signature absorption spectrum for an element or molecule. This absorption spectrum consists of dark absorption lines superimposed on a bright continuous spectrum. Each different element and molecule absorbs light at a unique set of frequencies producing a unique spectrum almost like a fingerprint. A hyperspectral scanner offers the capability to ‘see’ these unique fingerprints.

Using our invention, a hyperspectral scanner can be used to detect malformed proteins in the tissue of live or dead animals. This approach includes:

Use a hyperspectral retinal scanner to collect spectra of infected and non-infected animal tissue.
Classify unique spectral signatures for infected animals.
Determine an optimal algorithm to automatically identify these features in infected animals.
Validate the algorithm against a blind sample set.

Since one of the areas a BSE infection is most readily apparent is the eye tissue, this infestation will cause a spectral change to the tissue detectable via a hyperspectral retinal scanner. The resulting absorption feature will be indicative of the presence of BSE. This feature is likely caused by the prion proteins shown to be an indicator of BSE and/or the Cu+2 elevation caused by these same proteins. With the present invention one can characterize any absorption features unique to BSE and the infected tissue and develop an algorithm to automatically, non-invasively identify the disease. In so doing, it will be possible to develop tailored, low cost spectrometers able to detect the specific absorption features of target diseases (e.g., BSE) and an easy to understand manual for implementation by even field personnel like cattle handlers.

A hyperspectral retinal scanner offers this possibility of detecting the malformed prion proteins directly via accumulations of the malformed prions in eye tissue of live cattle. Some of the advantages of this approach are: no need to come in direct contact with the sample, no consumables (the system is like taking a digital picture), opportunity to detect other diseases from the same data (one scan previously detected 12 different proteins), no need to slaughter healthy animals, and the system can be fully automated for use by any cattle handler.

While the preferred embodiment is described in connection with a retinal scanner, other approaches for hyperspectral imaging of animal tissue for potential disease includes: using a stationary hyperspectral scanner with a scanning mirror attachment or a system whereby the hyperspectral scanner moves along a tracking mechanism across the object to be scanned.

### BRIEF DESCRIPTION OF THE DRAWINGS

FIG. 1 illustrates a hyperspectral imaging system using a retinal scanner that can embody the present invention.

FIG. 2 schematically illustrates functional blocks of embodiments of the present invention.

FIG. 3 shows an example scanner.

FIG. 4 schematically illustrates an example process for classifying spectra of interest.

FIG. 5 schematically illustrates an example process for operation of an imaging system in accordance with an embodiment of the present invention.

### DETAILED DESCRIPTION OF THE PREFERRED EMBODIMENTS

The following describes exemplary embodiments of the present invention and is presented only to illustrate some preferred embodiments of the present invention, not to limit the present invention to these exemplary embodiments.

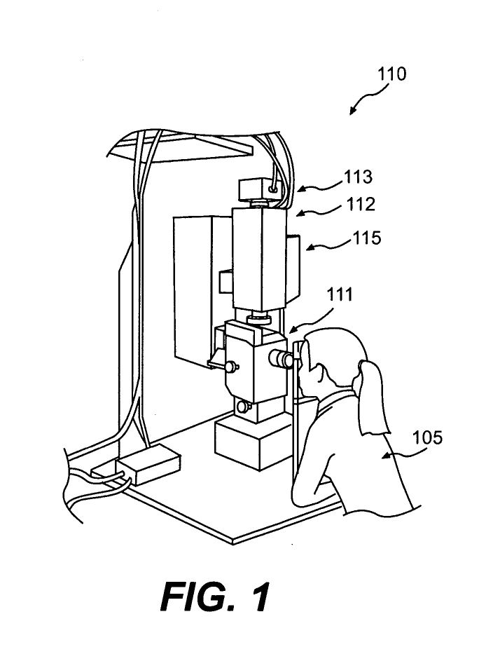

Turning first to a system for determining an optimal imaging system and algorithm, FIG. 1 illustrates a hyperspectral imaging system using a retinal scanner. A hyperspectral scanner 110 is used to obtain a hyperspectral image cube of the retinal region of a number of eyes. This scanner can be any available scanner, with appropriate calibrations, one such example being the Hyperspectral Fundus Imager currently available from Kestrel Corporation. This scanner 110 produces a high spectral resolution (3 to 5 nm) image for a single line across the patient's 105 retina, and uses a Fourier Transform imaging spectrometer 112 to preprocess the imaging data before capture by a CCD camera 113. The captured information is forwarded via coupler 114 to a data processing system (not shown).

A presently understood advantage of using a retinal image for the hyperspectral data is that next to the brain and spinal cord, the optical nerve is reported as the most likely location in the body to find TSE's (transmissible spongiform encephalopathy). However, since proteins, and thus prions, are detectible by hyperspectral imaging, in sufficient concentrations TSE's should similarly be detectible using other forms of hyperspectral imaging. This may include in vivo testing, as well as testing of tissue samples. An example of the latter includes a pre-imaging preparation such as electrophoresis gels to help spread out collected proteins and prions by an electric field, then imaging the sample via any suitable scanner. One such suitable scanner is a fixed table-top scanner with preset position for imaging the target samples, connected to a computer for processing the image data.

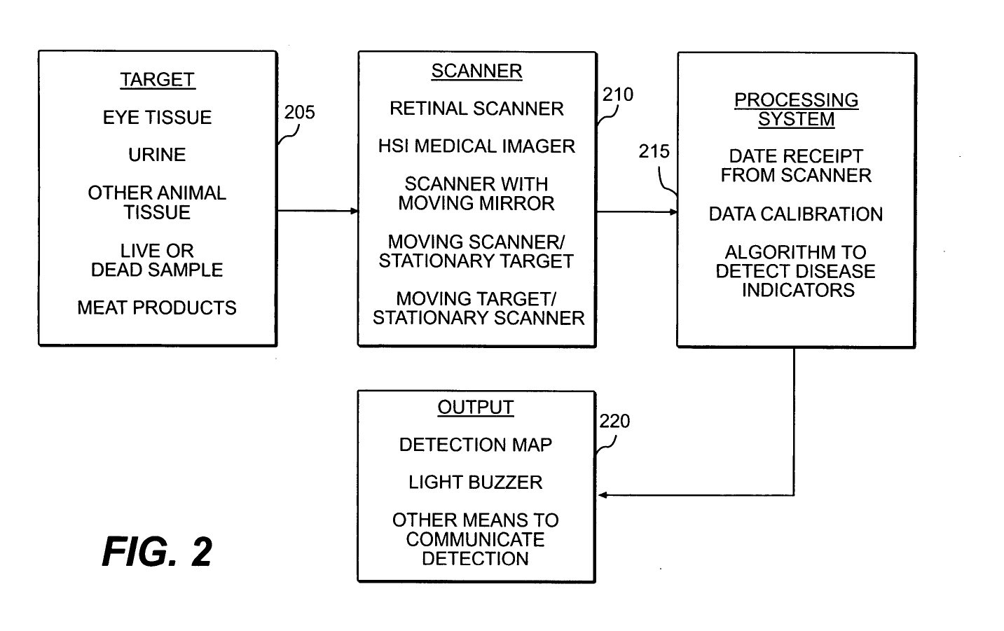

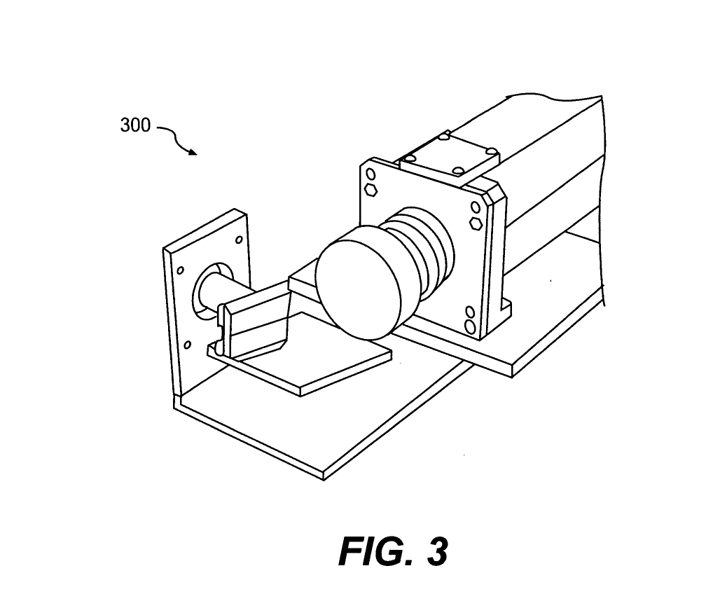

In its simplest form, an embodiment of the invention can include a target, a scanner, processing system and an output device. One example of an embodiment is shown in FIGS. 2 and 3. A target 205 is brought into alignment with an appropriate scanner 210 (examples of which include the scanner 110 of FIG. 1 and scanner 300 of FIG. 3), which outputs a predetermined form of data as a scan image file. The image data is then processed in a suitable information processing system 215, with the processed information output in a suitable detection format 220 (e.g., optical or audio alert, numeric value, etc.). FIG. 2 lists several common scan systems, along with examples of targets and processing systems. One skilled in the art will appreciate that these are merely illustrative of the types of scanners, targets, processing and output systems available, and that particular systems will vary based upon typical design choice criteria or routine experimental determination (e.g., testing different algorithms to determine which experimentally works best with a selected scanner system).

Referring to FIG. 2, the target 205 can be, for example, any animal tissue, any live or dead sample, eye tissue, urine, a meat product. The scanner 210 can be, for example, a retinal scanner, a HIS medical imager, a scanner with a moving mirror, a moving scanner with a stationary target, or stationary scanner with a moving target. Also as shown in the illustrative embodiment of FIG. 2, the processing system 215 receives and processes data based on hyperspectral imaging by the scanner 205. Obviously there does not need to be direct connection between the scanner 210 and the processing system 215; the data can be passed over a wired connection, a wireless connection, the internet, or mass storage device such a hard drive or CD or DVD or any other well known mechanism for transfer of data. The processing system 215 processes as discussed in the following. And, the output 220 shown in the exemplary embodiment of FIG. 2 can be a detection map, an audio output, a visual output, or any other indicator of detection (or non-detection).

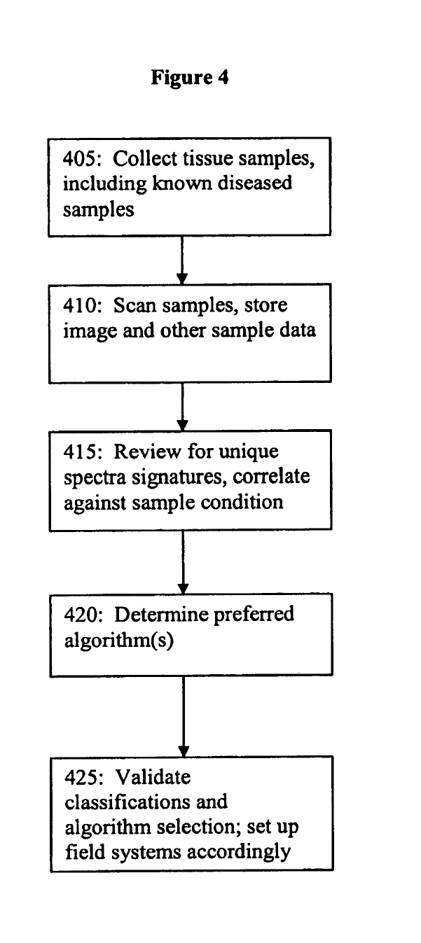

With reference now to FIG. 4, a process for classifying spectra of interest is shown. In the first step, tissue sample are mustered, including samples from known infected and non-infected animals. These samples preferably include intact eyeballs and/or live subjects, so the scanning includes samples taken under conditions approximating field conditions. While carefully tracking the known conditions relating to each sample, one or more scans are taken of each sample. When taking multiple scans of the same sample, one preferably captures a variety of information, which may include the size of the eye, portion of the retina scanned and entering at which point of the eye, special conditions (e.g., cataracts, floaters, etc.) and the like.

Once all scans are taken and associated with the pertinent sample data, the spectral scans are reviewed for unique spectral signatures associated with the animals, and in particular those unique signatures associated with the presence of TSE's. Based on these unique signatures, the next step is to determine an optimal algorithm to automatically identify these features in infected animals. These can be done by a study of the parameters associated with the unique signature and hyperspectral image, or by an iterative post-processing of the image information applying different candidate algorithms to determine which algorithm provides the best detection, or some combination of both. Those skilled in the art will readily understand how to determine the algorithm(s) to use in view of design choices such as the specific scanner used and the type of imaging being gathered.

In one illustrative process, spectra are compared from healthy and diseased samples. The diseased samples have preferably already been characterized by experts in the field as to the state of the infectivity of the animal. The spectral analyst will then analyze the spectra of the healthy and diseased animals, e.g., class one and two, to determine if there is a spectral correlation between healthy and infected animals, as well as feature depth correlation between animals in early stages of infection and those in latter stages of infection. One or more manual (i.e., visual inspection by analysts) and automatic (e.g., commercially available software such as BandMax™) are then used to identify spectral differences between the two classes and/or identify locations of spectral contrast. The analyst (or program) upon determining the spectral contrast points can then couple a commercially available and/or new algorithm(s) to maximize the ability to automatically identify the features associated with the disease(s) of interest. For example if specific, unique features indicate presence of the disease then an algorithm based upon spectral angle might be used; if the presence of disease is determined via a spectral slope change then a matched filter approach might be applied.

Finally, after identifying the target signatures and optimal algorithms, the classifications and choices are preferably confirmed via a validation step. This step may be implemented by an appropriate validation system, but is typically accomplished by setting up a standard scanner implementation and operating it with the selected algorithm against a blind sample set under anticipated field conditions.

Turning now to FIG. 5, a example process for operation of an imaging system is shown. In the first step, the target tissue is aligned with a scanner, and one or more hyperspectral images taken of the region of interest. In the preferred operation, the region of interest is the retina of an animal, with the images being taken via a retinal scanner through the lens of a live animal. The retina provides imaging of both exposed nerve tissue and blood vessels, and depending on the image being collected, can view signatures based on TSE's themselves (e.g., in the retinal nerves) or telltale byproducts (e.g., in the blood).

In a preferred embodiment, the collected images are contemporaneously processed with the algorithm or algorithms of choice, so an immediate determination can be made to study or isolate animals testing positive for TSEs. One may also want to forward the data collected for remote processing and evaluation, or merely for storage and further studying at a latter date. Given the advances in hyperspectral processing, it is possible to use a post processing stage to both validate field tests against the selected algorithms, but also to run further tests with additional algorithms.

Finally, given the unique characteristics of animal retinas, it is also possible to capture a sufficiently detailed identifying image of each retina tested, so the digital hyperspectral image is associated with sufficient digital identifying information to uniquely associate a set of images with the tested animal. This is advantageous, e.g., in preventing a mis-identification of an image testing positive with the wrong animal; it may also be useful in large field tests, in helping to identify animals from similar lots or, if an animal's other identification tags have been misapplied, in locating a particular animal again.

Those skilled in the art will appreciate that there are numerous benefits from our novel process. Among these are:
the potential to identify malformed prion proteins directly
the potential to perform tests for TSEs and proteins on live animals, in an efficient and cost-effective process
the potential for real time detection of TSEs and proteins
the potential to implement in single systems, capable of field operation, without the need for routine consumables
no requirement to handle hazardous materials (since even infected animals are tested live without contact with body fluids, etc.)
the potential for early detection of diseases, including BSE
the potential to detect multiple disease from the same collected data
the potential to detect human version of BSE, Cruetzfeldt-Jakob Disease
the potential to detect other human heath issue (SARS was recently detectable in human tears, so other diseases present in any location that is scannable can be detected—and the scanner can be external (such as a retinal scanner) or even internal, if mounted on any of the variety of scopes used for internal procedures).

What is claimed is:

1. A hyperspectral imaging method for distinguishing between normal and abnormal tissue, comprising:
a. receiving information based on hyperspectral imaging of tissue;
b. providing information representative of an identifiable spectral feature;
c. determining if said information includes data representative of said identifiable spectral feature; and
d. providing an indicator based on said results of said determining.

2. A hyperspectral imaging method according to claim 1, wherein said determining includes recognizing whether said tissue corresponds to normal or abnormal tissue.

3. A hyperspectral imaging method according to claim 1, wherein said hyperspectral imaging includes retinal scanning.

4. A hyperspectral imaging method according to claim 1, wherein said hyperspectral imaging includes scanning of live tissue.

5. A hyperspectral imaging method according to claim 1, wherein said hyperspectral imaging includes scanning of a dead tissue sample.

6. A hyperspectral imaging method according to claim 1, wherein said hyperspectral imaging includes providing a detection map.

7. A hyperspectral imaging system to distinguish between normal and abnormal tissue, comprising:
a. a hyperspectral scanner for scanning tissue and providing information indicative of the scanned tissue;
b. a processing system operatively connected to receive data based on scanning by the hyperspectral scanner so as to determine if the received data includes data representative of an identifiable spectral feature; and
c. an indicator responsive to said processing system determining if the received data includes data representative of an identifiable spectral feature.

8. A hyperspectral imaging system according to claim 7, wherein said processing system includes processing to recognize whether said tissue corresponds to normal or abnormal tissue.

9. A hyperspectral imaging system according to claim 7, wherein said hyperspectral scanner includes a retinal scanner.

10. A hyperspectral imaging system according to claim 7, wherein said hyperspectral scanner includes a scanner capable of scanning at least one of live tissue and dead tissue.

11. A hyperspectral imaging system according to claim 7, wherein said indicator includes a detection map.

12. A hyperspectral imaging system according to claim 7, wherein said indicator includes at least one of an audio indicator and a visual indicator.
